(19) United States
(12) Patent Application Publication
(10) Pub. No.: US 2012/0253170 A1
(43) Pub. Date: Oct. 4, 2012

(54) METHOD AND APPARATUS FOR GENERATING MEDICAL IMAGE OF BODY ORGAN BY USING 3-D MODEL

(75) Inventors: Jung-bae Kim, Hwaseong-si (KR); Won-chul BANG, Seongnam-si (KR); Young-kyoo HWANG, Seoul (KR); Yong-sun KIM, Yongin-si (KR)

(73) Assignee: Samsung Electronics Co., Ltd., Suwon-si (KR)

(21) Appl. No.: 13/425,597

(22) Filed: Mar. 21, 2012

Related U.S. Application Data
(60) Provisional application No. 61/468,754, filed on Mar. 29, 2011.

(30) Foreign Application Priority Data
Aug. 29, 2011 (KR) 10-2011-0086694

(51) Int. Cl. A61B 5/055; G06T 17/00; G06K 9/00; A61B 8/00

(52) U.S. Cl. 600/410; 600/437; 345/419; 382/131

(57) ABSTRACT

A method of generating an image of an organ includes generating a three-dimensional (3-D) model of at least one organ of a patient based on a medical image of the at least one organ; generating a plurality of matched images by matching a plurality of images showing a change of a shape of the at least one organ due to a body activity of the patient to the 3-D model of the at least one organ; selecting one of the plurality of matched images based on a current body condition of the patient; and outputting the selected matched image.

### CROSS-REFERENCE TO RELATED APPLICATIONS

This application claims the benefit of U.S. Provisional Application No. 61/468,754 filed on Mar. 29, 2011, and Korean Patent Application No. 10-2011-0086694 filed on Aug. 29, 2011, in the Korean Intellectual Property Office, the disclosures of which are incorporated herein by reference in their entirety.

### BACKGROUND

1. Field

This disclosure relates to a method and an apparatus for generating a medical image of a body organ by using a three-dimensional (3-D) model.

2. Description of the Related Art

In traditional methods of diagnosing and treating diseases, a state of a disease is confirmed with the naked eye after performing a laparotomy, and then an incision or plastic surgery is performed on a lesion with the use of large surgical instruments. However, recently, a method of treating diseases without making an incision in the body of a patient has been developed since it has become possible to obtain high resolution medical images and also to minutely control a medical instrument due to the progress of medical technology.

In this method, treatment of a disease is performed while directly inserting a catheter or a medical needle into a blood vessel or a body part after making a small hole in the skin of the patient, and then observing the inside of the body of the patient by using a medical imaging apparatus. This method is referred to as a “surgical operation using image,” an “interventional image-based surgical operation,” or a “mediate image-based surgical operation.”

In this method, a surgeon determines a location of an internal organ or a lesion through images. Moreover, the surgeon needs to be aware of any change due to the patient's breathing or movement during a surgical operation. Thus, the surgeon needs to accurately and rapidly determine the patient's breathing or movement based on real time images to perform the surgical operation, but it is not easy to determine a shape of the internal organ or the lesion with the naked eye. Thus, in order to solve this problem, methods and apparatuses for allowing the surgeon to determine a shape and a location of an internal organ in real time have been developed.

### SUMMARY

According to an aspect, a method of generating an image of an organ includes generating a three-dimensional (3-D) model of at least one organ of a patient based on a medical image of the at least one organ; generating a plurality of matched images by matching a plurality of images showing a change of a shape of the at least one organ due to a body activity of the patient to the 3-D model of the at least one organ; selecting one of the plurality of matched images based on a current body condition of the patient; and outputting the selected matched image.

The generating of the 3-D model may include generating the 3-D model to show the shape of the at least one organ of the patient based on the medical image of the at least one organ.

The selecting may include selecting one of the plurality of matched images based a real time medical image showing the current body condition of the patient; and the plurality of images and the real time medical image may be ultrasound images.

The generating of the plurality of matched images may include modifying the 3-D model based on the change of the shape of the at least one organ; and fitting a coordinate axis of the 3-D model to a coordinate axis of the plurality of images.

The generating of the plurality of matched images may further include generating the plurality of matched images by overlapping pixel or voxel values of the plurality of images with a predetermined brightness.

The selecting may include selecting one of the plurality of matched images corresponding to one of the plurality of images that is most similar to a real time medical image of the patient showing the current body condition of the patient.

The selecting may include calculating a difference between a location of a diaphragm in each of the plurality of images and a location of a diaphragm in the real time medical image; and selecting one of the plurality of matched images that corresponds to one of the plurality of images for which the calculated difference is the smallest among all of the plurality of matched images.

The generating of the 3-D model may include extracting location coordinate information of a boundary and an internal structure of the at least one organ from the medical image; designating coordinates of landmark points in the location coordinate information; and generating a statistical external appearance 3-D model of the at least one organ based on the coordinates of the landmark points.

The generating of the 3-D model may further include changing the statistical external appearance 3-D model to a 3-D model reflecting a shape characteristic of the at least one organ of the patient.

The generating of the 3-D model may further include reflecting the shape characteristic of the at least one organ of the patient onto the medical image of the at least one organ.

The shape characteristic may include a shape and a location of a lesion of the at least one organ.

The extracting of the location coordinate information may include determining a position in the medical image at which a change in a brightness value is a maximum as the location coordinate information of the boundary and the internal structure of the at least one organ.

The extracting of the location coordinate information may include determining a position in the medical image at which a frequency value of a discrete time Fourier transform (DTFT) is a maximum as the location coordinate information of the boundary and the internal structure of the at least one organ.

The extracting of the location coordinate information may include determining the location coordinate information of the boundary and the internal structure of the at least one organ based on coordinates input by a user.

The plurality of images may be images captured at predetermined intervals during a breathing cycle of the patient.

The medical image of the at least one organ may be an image captured using a computed tomography (CT) method.

The medical image of the at least one organ may be an image captured using a magnetic resonance (MR) method.

The generating of the 3-D model may include pre-generating the 3-D model prior to beginning preparations to treat the patient; storing the pre-generated 3-D model in a database prior to beginning the preparations to treat the patient; and retrieving the pre-generated 3-D model stored in the database as part of the preparations to treat the patient.

According to an aspect, a non-transitory computer-readable storage medium may store a program for controlling a processor to perform a method of generating an image of an organ as described above.

According to an aspect, an apparatus for generating an image of an organ includes an organ model generation unit configured to generate a 3-D model of at least one organ of a patient based on a medical image of the at least one organ; an image matching unit configured to generate a plurality of matched images by matching a plurality of images showing a change of a shape of the at least one organ due to a body activity of the patient to the 3-D model of the at least one organ; and an image search unit configured to select one of the plurality of matched images based on a current body condition of the patient, and output the selected matched image.

The apparatus may include an additional adjustment unit configured to further adjust the plurality of matched images according to an input from a user.

According to an aspect, a method of generating an image of an organ includes generating an average three-dimensional (3-D) model of an organ based on a plurality of medical images of the organ; generating a private 3-D model of the organ in a specific patient based on the average 3-D model of the organ and at least one medical image of the organ of the patient; generating a plurality of matched images by matching a plurality of images of the organ of the patient in which a shape of the organ of the patient changes due to a body activity of the patient to the private 3-D model of the organ; selecting one of the matched images based on a real time medical image of the organ of the patient reflecting a current body condition of the patient; and outputting the selected matched image.

The selecting may include selecting one of the matched images that corresponds to one of the plurality of images of the organ of the patient in which a location and/or a shape of the organ of the patient is most similar to a location and/or a shape of the organ of the patient in the real-time medical image of the organ of the patient.

The plurality of medical images of the organ and the medical image of the organ of the patient may be computed tomography (CT) images or magnetic resonance (MR) images; and the plurality of images of the organ of the patient and the real time medical image of the organ of the patient may be ultrasound images.

The body activity of the patient may be breathing; and the plurality of images of the organ of the patient may be captured at predetermined intervals during one complete breathing cycle of the patient.

By using the method and the apparatus, it is possible to accurately and rapidly track a location of an organ during a surgical operation by combining a real time medical image with a graphical model of the organ and outputting a combined image.

Additional aspects will be set forth in part in the description that follows, and, in part, will be apparent from the description, or may be learned by practice of the described examples.

### BRIEF DESCRIPTION OF THE DRAWINGS

The above and/or other aspects will become apparent and more readily appreciated from the following description of examples, taken in conjunction with the accompanying drawings of which:

FIG. 1 is a diagram illustrating a configuration of a system for generating an image of a body organ according to an example of the invention;

FIG. 2 is a block diagram illustrating a configuration of an image matching device of FIG. 1;

FIG. 3 is a diagram for explaining a process of extracting location coordinate information of a boundary and an internal structure of an organ from external medical images;

FIG. 4 is a flowchart illustrating a process in which an image matching unit of FIG. 2 fits a private 3-D body organ model modified to reflect a change in an organ to a location of the organ in each of a plurality of ultrasound images;

FIG. 5 illustrates a process of applying an affine transformation function in a two-dimensional (2-D) image;

FIG. 6 illustrates a process of matching images performed by the image matching unit of FIG. 2;

FIG. 7 is a graph illustrating an up and down movement of an absolute location of a diaphragm; and

FIG. 8 is a flowchart illustrating a method of tracking a dynamic organ and a lesion based on a three-dimensional (3-D) body organ model.

### DETAILED DESCRIPTION

Examples of the invention will now be described more fully with reference to the accompanying drawings. In the following description, well-known functions or constructions will not be described in detail to avoid obscuring the invention with unnecessary detail.

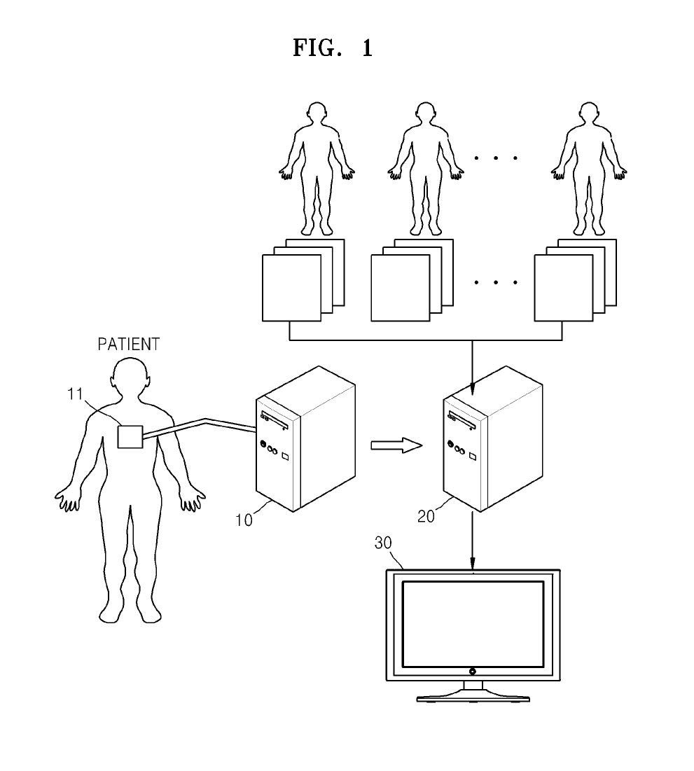

FIG. 1 is a diagram illustrating a configuration of a system for generating an image of a body organ according to an example of the invention. Referring to FIG. 1, the system includes an image detection device 10, an image matching device 20, and an image display device 30. The image detection device 10 generates image data by using a response that is generated by transmitting a source signal generated from a probe 11 of the image detection device 10 to a target part of a patient's body. The source signal may be a signal such as an ultrasound signal, an X-ray, or the like. An example in which the image detection device 10 is an ultrasonography machine that captures three-dimensional (3-D) images of the patient's body by using ultrasound is described below.

In the ultrasonography machine, the probe 11 is generally in the form of a piezoelectric transducer. If an ultrasound signal in the range of 2 to 18 MHz is transmitted from the probe 11 of the image detection device 10 to a part of the inside of the patient's body, the ultrasound signal will be partially reflected from layers between various different tissues. In particular, the ultrasound will be reflected from parts where a density changes in the inside of the body, for example, blood cells of blood plasma, small structures of organs, and the like. The reflected ultrasound vibrates the piezoelectric transducer of the probe 11, and the piezoelectric transducer outputs electrical pulses due to the vibration. The electrical pulses are converted into images by the image detection device 10.

The image detection device 10 may output two-dimensional (2-D) images and may also output 3-D images. A method in which the image detection device 10 outputs 3-D images is as follows. The image detection device 10 captures a plurality of cross-sectional images of a part of the patient's body while changing a location and an orientation of the probe 11 over the patient's body. The image detection device 10 accumulates the cross-sectional images and generates 3-D volume image data indicating three-dimensionally the part of the patient's body from the cross-sectional images. In this manner, a method of generating the 3-D volume image data by accumulating the cross-sectional images is referred to as multi-planar reconstruction (MPR) method.

However, although images obtained by the image detection device 10, for example, ultrasound images, may be obtained in real time, it is difficult to clearly identify a boundary and an internal structure of an organ or a lesion through the ultrasound images.

In computed tomography (CT) images or magnetic resonance (MR) images, a location of an organ or a lesion may be clearly identified. However, when the patient breathes or moves during a surgical operation, the shape of the organ or the lesion may be transformed or the location of the organ or the lesion may be changed, and an image reflecting the real time change may be not obtained by using the CT images or MR images. That is, the CT images may not be output in real time because the CT images are obtained by using radiation and thus require short time photographing due to a danger to the patient or surgeon of prolonged radiation exposure. The MR images may not be output in real time because it takes a long time to capture them.

Thus, it is necessary to provide a method and apparatus that may capture images in real time and also clearly identify a boundary and an internal structure of an organ or a lesion. Thus, examples that will be explained below provide a method in which a location or a transformation of an organ or a lesion may be correctly identified by outputting images in which images detected in real time are matched to a model of an organ or a lesion.

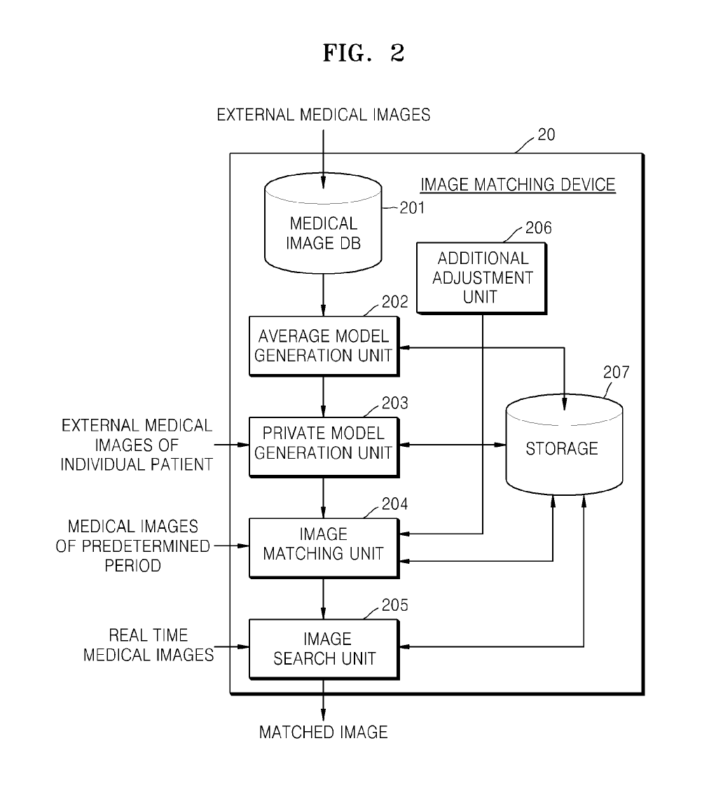

FIG. 2 is a block diagram illustrating a configuration of the image matching device 20 of FIG. 1. Referring to FIG. 2, the image matching device 20 includes a medical image database (DB) 201, an average model generation unit 202, a private model generation unit 203, an image matching unit 204, an image search unit 205, an additional adjustment unit 206, and a storage 207. The various units 202, 203, 204, 205, and 206 that are described in detail below may be implemented as hardware components, software components, or components that are a combination of hardware and software.

The average model generation unit 202 generates an average model of an organ by receiving various medical images of a patient and then processing them. In this example, an organ of a patient is tracked by using a private model, i.e., a personalized model of the patient. The average model is generated by the average model generation unit 202 as a preparatory step for generating the private model. This is because, since characteristics of an organ, such as a shape and a size, are different for each individual person, it is necessary to reflect the characteristics of each individual to provide an accurate surgical operation environment. Various pieces of image information of each individual may be used to obtain an accurate average model. In addition, images at various points of breathing may be obtained to reflect a form of an organ that changes according to the breathing.

In greater detail, the average model generation unit 202 receives images (hereinafter referred to as “external medical images”) that a medical expert has captured for diagnosis of a patient, directly from a photographing apparatus or from an image storage medium. Thus, it is desirable to receive external medical images that make it possible to easily analyze boundaries of an organ or a lesion or characteristics of the inside of the organ. For example, CT images or MR images may be input as the external medical images.

The external medical images are stored in the medical image DB 201, and the average model generation unit 202 receives the external medical images stored in the medical image DB 201. The medical image DB 201 may store medical images of various individuals that may be captured by the photographing apparatus or may be input from the image storage medium. When receiving the external medical images from the medical image DB 201, the average model generation unit 202 may receive some or all of the external medical images from the medical image DB 201 depending on a selection of a user.

The average model generation unit 202 applies a 3-D active shape model (ASM) algorithm to the received external medical images. In order to apply the 3-D ASM algorithm, the average model generation unit 202 extracts a shape, a size, and anatomic features of an organ from the received external medical images by analyzing the received external medical images, and generates an average model of the organ by averaging them. The 3-D ASM algorithm is described in detail in the paper “The Use of Active Shape Models For Locating Structures in Medical Images,” Image and Vision Computing, Vol. 12, No. 6, July 1994, pp. 355-366, by T. F. Cootes, A. Hill, C. J. Taylor, and J. Haslam, which is incorporated herein by reference in its entirety. It is possible to obtain an average shape of the organ by applying the 3-D ASM algorithm, and the average shape of the organ may be transformed by modifying variables.

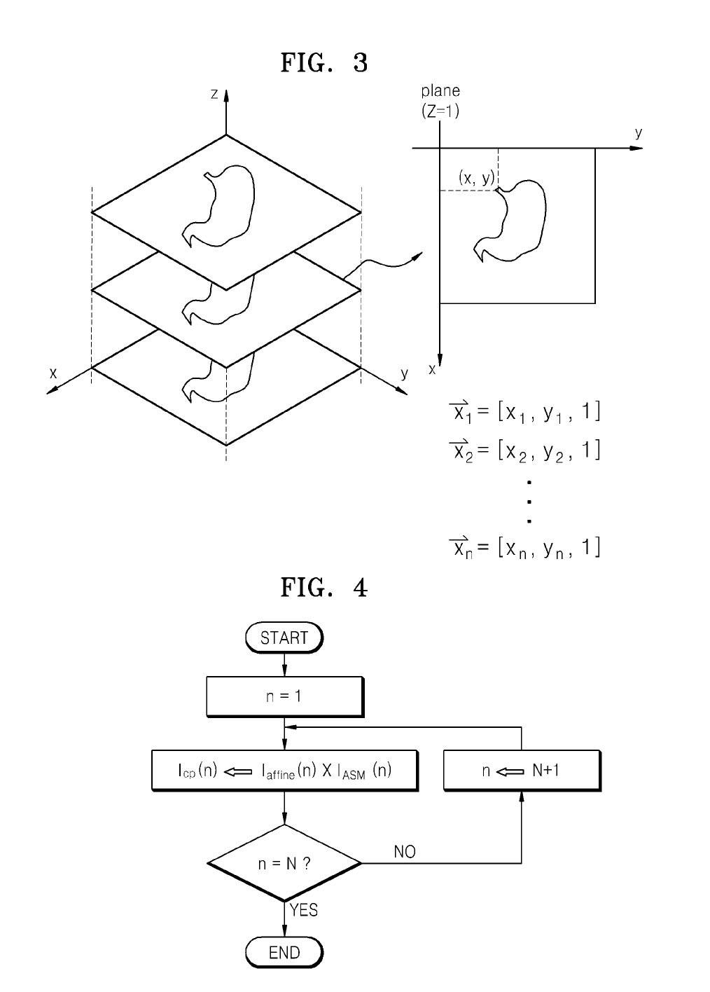

FIG. 3 is a diagram for explaining a process extracting location coordinate information of a boundary and an internal structure of an organ from the external medical images, for example, the CT or MR images. For example, an internal structure of a liver may include a hepatic artery, a hepatic vein, a hepatic duct, and boundaries between them. When the external medical images are input to the average model generation unit 202, the average model generation unit 202 performs an operation of extracting the location coordinate information of the boundary and the internal structure of the organ by using different methods depending on whether the external medical images are 2-D images or 3-D images.

If 2-D images are input as the external medical images, the average model generation unit 202 obtains a 3-D volume image indicating three-dimensionally a target part by accumulating a plurality of cross-sectional images to generate a 3-D model. This method of obtaining the 3-D volume image is illustrated in the left side of FIG. 3. In more detail, before accumulating the plurality of cross-sectional images, the location coordinate information of the boundary and the internal structure of the organ is extracted from each of the plurality of cross-sectional images. Then, it is possible to obtain 3-D coordinate information by adding coordinate information of an axis of a direction in which the plurality of cross-sectional images are accumulated to the extracted information. For example, since the image illustrated in the right side of FIG. 3 is an image whose Z-axis value is 1, a Z value of a location coordinate of a boundary extracted from the image is always 1. That is, 3-D coordinate information of the image illustrated in the right side of FIG. 3 is [x,y,1]. Thus, since coordinate information of cross-sectional images illustrated in the left side of FIG. 3 is 2-D coordinate information [x,y], both a coordinate value of the Z-axis and the 2-D coordinate information [X,Y] are extracted to obtain the location coordinate information of the images illustrated in the left side of FIG. 3. Then, the location coordinate information of the images will be 3-D coordinate information [x,y,z].

If 3-D images are input as the external medical images, cross-sections of the 3-D images are extracted at predetermined intervals to obtain cross-sectional images, and then the same process as the case where 2-D images are input as the external medical images is performed, thereby obtaining 3-D location coordinate information.

In this process, location coordinate information of a boundary of an organ in 2-D images may be automatically or semi-automatically obtained by using an algorithm, and may also be manually input by a user with reference to output image information.

For example, in a method of automatically obtaining the location coordinate information of the boundary of the organ, it is possible to obtain location coordinate information of a part in which a brightness of an image is abruptly changed, and it is also possible to extract a location at which a frequency value is largest as a boundary location by using a discrete time Fourier transform (DTFT).

In a method of semi-automatically obtaining the location coordinate information of the boundary of the organ, if information about a boundary point of an image is input by a user, it is possible to extract the location coordinate of a boundary based on the boundary point, similar to the method of automatically obtaining the location coordinate information. Since the boundary of the organ is continuous and has a looped curve shape, it is possible to obtain information about the whole boundary of the organ by using this characteristic. Since the method of semi-automatically obtaining the location coordinate information does not require searching for the whole of an image, it is possible to rapidly obtain a result compared to the method of automatically obtaining the location coordinate information.

In a method of manually obtaining the location coordinate information of the boundary of the organ, a user may directly designate coordinates of a boundary while viewing the image. At this time, since an interval at which the coordinates of the boundary is designated may not be continuous, it is possible to continuously extract the boundary by performing interpolation with respect to discontinuous sections. If the location coordinate information of the organ or a lesion obtained by using the above methods is output after setting a brightness value of a voxel corresponding to the location coordinate to a predetermined value, the user may confirm shapes of the organ or the lesion expressed three-dimensionally and graphically. For example, if a brightness value of boundary coordinates of a target organ is set to a minimum value, namely the darkest value, an image of the target organ will have a dark form in an output image. If the brightness value of the target organ is set to a medium value between a white color and a black color and the brightness value of a lesion is set to the black color, it is possible to easily distinguish the lesion from the target organ with the naked eye. The location coordinate information of boundaries and internal structures of a plurality of organs, obtained by using the above methods, may be defined as a data set and may be used to perform the 3-D ASM algorithm. The 3-D ASM algorithm is explained below.

In order to apply the 3-D ASM algorithm, coordinate axes of location coordinates of the boundaries and the internal structures of the plurality of organs are fit to each other. Fitting the coordinate axes to each other means fitting the centers of gravities of the plurality of organs to one origin and aligning directions of the plurality of organs. Thereafter, landmark points are determined in the location coordinate information of the boundaries and the internal structures of the plurality of organs. The landmark points are basic points used to apply the 3-D ASM algorithm. The landmark points are determined by using following method.

First, points in which a characteristic of a target is distinctly reflected are determined as landmark points. For example, the points may include division points of blood vessels of a liver, a boundary between the right atrium and the left atrium in a heart, a boundary between a main vein and an outer wall of the heart, and the like.

Second, the highest points or the lowest points of a target in a predetermined coordinate system are determined as landmark points.

Third, points for interpolating between the first determined points and the second determined points are determined as landmark points along a boundary at predetermined intervals.

The determined landmark points may be represented by using coordinates of the X and Y axes in two dimensions, and may be represented by using coordinates of the X, Y, and Z axes in three dimensions. Thus, if coordinates of each of the landmark points are indicated as vectors x₀, x₁, . . . xₙ₋₁ in three dimensions (where n is the number of landmark points), the vectors x₀, x₁, . . . xₙ₋₁ may be represented by the following Equation 1:

$${x}_{i 0} [ {x}_{i 0} , {y}_{i 0} , {z}_{i 0} ] \\ {x}_{i 1} [ {x}_{i 1} , {y}_{i 1} , {z}_{i 1} ] \\ ⋮ \\ {x}_{in - 1} = [ {x}_{i n - 1 } , {y}_{in - 1} , {z}_{i n - 1} ] \qquad ( 1 )$$

The subscript i indicates location coordinate information of a boundary and an internal structure of an organ obtained in an i-th image. The number of the location coordinate information may be increased in some cases, and thus, the location coordinate information may be represented as a single vector to facilitate calculation. Then, a landmark point vector that expresses all of the landmark points with a single vector may be defined by the following Equation 2:

xᵢ=[xᵢ₀, yᵢ₀, zᵢ₀, xᵢ₁, yᵢ₁, zᵢ₁, . . . , xᵢₙ₋₁, yᵢₙ₋₁, zᵢₙ₋₁]^{T} (2)

The size of the vector xᵢ is 3n×1. If the number of images in the data set is N, an average of the landmark points for all of the images in the data set may be represented by the following Equation 3:

$$\overline{x} = \frac{1}{N} {\sum }_{i = 1}^{N} {x}_{i} \qquad ( 3 )$$

The size of the vector x̅is 3n×1. The average model generation unit 202 obtains the average x̅ of the landmark points using Equation 3, generates a model based on the average x̅ of the landmark points, which becomes an average organ model. The 3-D ASM algorithm not only may generate the average organ model, but may also change only a form of the average organ model by adjusting a plurality of parameters. Thus, the average model generation unit 202 calculates not only the average organ model but also uses an equation so that the plurality of parameters may be applied. An equation for applying the plurality of parameters will be explained below.

A difference between the landmark points xᵢ and the average x̅ of the landmark points may be represented by the following Equation 4. In Equation 4, the subscript i indicates an i-th image. Thus, Equation 4 indicates a difference between the landmark points xᵢ of each image i and the average x̅ of the landmark points of all of the images in the data set.

dxᵢ=xᵢ− x̅ (4)

Based on the difference dxᵢ, a covariance matrix for three variables x, y, and z may be defined by the following Equation 5. The reason for obtaining the covariance matrix is to obtain a unit eigenvector for the plurality of parameters to apply the 3-D ASM algorithm.

$$S = \frac{1}{N} {\sum }_{i = 1}^{N} {dx}_{i} {dx}_{i}^{T} \qquad ( 5 )$$

The size of the covariance matrix S is 3n×3n . If the unit eigenvectors of the covariance matrix S are pₖ (k=1, 2, . . . 3n), the unit eigenvector pₖ indicates a change of a model generated by using the 3-D ASM algorithm. For example, if a parameter b₁ multiplying a unit eigenvector p₁ is changed within a range of −2\sqrt{λ₁}≦b₁<2\sqrt{λ₁} (λ₁ will be defined below), a width of the model may be changed. If a parameter b₂ multiplying a unit eigenvector p₂ is changed within a range of −2\sqrt{λ₂}≦b₂<2\sqrt{λ₂} (λ₂ will be defined below), a height of the model may be changed. The unit eigenvectors pₖ having a size 3n×1 may be obtained from the following Equation 6:

Spₖ=λₖ pₖ (6)

In Equation 6, λₖ is the k-th eigenvalue of S, where λₖ≧λₖ₊₁.

Finally, the landmark point vector x to which the change of the model is applied may be calculated by using the average vector x̅ of the landmark points as in the following Equation 7.

x= x̅+Pb (7)

P=(p₁, p₂, . . . pₜ) indicates t unit eigenvectors (here, the size of pₖ is 3n×1, and the size of P is 3n×t), b=(b₁, b₂ . . . bₜ)^{T} is a vector of weights, one for each of the t unit eigenvectors (here, the size of b is t×1).

The average model generation unit 202 calculates x̅ (the size thereof is 3n×1), which indicates a form of an average organ model, and the vector P=(p₁, p₂, . . . pₜ) (the size thereof is 3n×t), which is used to apply the change of the model by using the 3-D ASM algorithm by using the equations.

The private model generation unit 203 receives the average organ model x̅ and the vector P=(p₁, p₂, . . . pₜ) from the average model generation unit 202 and then generates a private model through parameter processing of the 3-D ASM algorithm. Since shapes and sizes of organs of patients are different according to the individual patient, accuracy may be lowered if the average organ model is used as it is. For example, an organ of a patient may have a longer, wider, thicker, or thinner form compared to organs of other patients. In addition, if an organ of a patient includes a lesion, the private model generation unit 203 may include a location of the lesion in a model of the organ to accurately capture a shape and a location of the lesion. Thus, the private model generation unit 203 receives external medical images of the individual patient from an external image photographing apparatus or the storage 207, analyzes a shape, a size, and a location of an organ of the individual patient, and if there is a lesion, analyzes a shape, a size, and a location of the lesion. The operation of the private model generation unit 203 is explained below with respect to an organ, but the same procedure can be used with respect to a lesion.

The private model generation unit 203 determines weights (the vector b) of the unit eigenvectors of the 3-D ASM algorithm for the individual patient based on the medical images, such as the CT or MR images, in which a shape, a size, and a location of an organ may be clearly captured. Thus, first, the private model generation unit 203 receives the external medical images of the individual patient and obtains location coordinate information of a boundary and an internal structure of an organ. In order to obtain the location coordinate information of the boundary and the internal structure of the organ, the private model generation unit 203 uses the process of FIG. 3, namely the process of analyzing the external medical images, that is performed by the average model generation unit 202. Furthermore, by determining coordinate information of the landmark points through a method that is the same as that used when applying the 3-D ASM algorithm, it is possible to obtain the vector x (the size thereof is 3n×1), which is a private landmark point set of the individual patient. An organ model generated based on the vector x may be a private model. If a characteristic (pₗ^{T} pₖ=1) of an inverse function and a unit eigenvector is used in Equation 7, the following Equation 8 may be obtained. A value of b=(b₁, b₂ . . . bₜ)^{T} is determined by Equation 8.

b=P^{T} (x− x̅) (8)

The vectors x̅ and P determined by the average model generation unit 202 may be stored in the storage 207 as a database of an average model for a target organ, and may be repeatedly used if necessary. In addition, the external medical images of the individual patient that are input to the private model generation unit 202 may be additionally used when determining the average model stored in the database during a medical examination and a treatment of another patient.

When the image matching unit 204 receives the vectors x, x̅,P,b from the private model generation unit 203, the image matching unit 204 matches the vectors with a patient's medical images received during a predetermined period. This matching means that a model obtained using the 3-D ASM algorithm is overlapped with a location of an organ in an ultrasound medical image to output an output image. In greater detail, the matching means that it is possible to replace or overlap pixel or voxel values corresponding to coordinate information of a model obtained using the 3-D ASM algorithm with a predetermined brightness. If the replacement operation is performed, an organ part is removed from an original ultrasound medical image and only a private model is output. If the overlap operation is performed, an image in which the original ultrasound medical image is overlapped with the private model may be output. The overlapped image may be easily identified with the naked eye by differentiating a color thereof from that of another image. For example, it may be easy to identify a graphic figure with the naked eye by overlapping a private model with a black and white ultrasound image by using a blue color.

The medical images may be images captured in real time and, for example, may be ultrasound images. The medical images may be 2-D or 3-D images. The predetermined period may be one breathing cycle. This is because a change of an organ also is generated during a breathing cycle of the body. For example, if one breathing cycle of a patient is 5 seconds, ultrasound images having 100 frames may be generated during one breathing cycle when ultrasound images are generated at 20 frames per second.

A process of matching that is performed in the image matching unit 204 may be divided into two operations. The two operations include an operation of modifying a 3-D body organ model to reflect a change of an organ due to breathing in ultrasound images input during a predetermined period, and an operation of aligning the modified 3-D body organ model to a target organ in the ultrasound images by performing rotation, parallel displacement, and scale control.

The operation of reflecting a change of an organ due to breathing to a 3-D body organ model is as follows. Before matching the ultrasound images with medical images, a value of the vector b, which is a weight for each unit eigenvector of the 3-D ASM algorithm, is controlled by obtaining a location and a change of an organ for each frame of the ultrasound images. A value of the vector b determined at this time does not have a large difference from a value of the vector b determined in the average model generation unit 202. This is because only a change due to the breathing is reflected in the image matching unit 204, and this change due to the breathing is small compared to changes in other individuals. Thus, when determining the value of the vector b, a modification is performed within a predetermined limited range based on the value of the vector b determined in the average model generation unit 202. In addition, a vector b of a previous frame may be reflected in a determination of a vector b of a next frame. This is because there is no large change during a short period between frames since a change of an organ during the breathing is continuous. If the value of the vector b is determined, it is possible to generate a private model for each frame in which a modification of an organ is reflected in each ultrasound image by using a calculation of the 3-D ASM algorithm.

FIG. 4 is a flowchart illustrating a process in which the image matching unit 204 fits a private 3-D body organ model modified to reflect a change in an organ to a location of the organ in each of a plurality of ultrasound images through rotation, parallel displacement, and scale control. In greater detail, FIG. 4 is a flowchart illustrating a process of performing one-to-one affine registration for each frame when the vector b, which is a weight of each unit eigenvector for each frame, is determined. If the number of frames is N and n is a frame number, a one-to-one matching is performed from n=1 to n=N. An affine transformation function is obtained by performing an iterative closest point (ICP) algorithm for each frame by using a landmark point set of an ultrasound image and a landmark point set of a model, and a 3-D body organ model image is obtained by using the affine transformation function. The ICP algorithm is an algorithm for performing rotation, parallel displacement, and scale control of other images based on an image to align a target in a plurality of images. The ICP algorithm is described in detail in “Iterative Point Matching for Registration of Free-form Curves and Surfaces,” International Journal of Computer Vision, Vol. 13, No. 2, October 1994, pp. 119-152, by Zhengyou Zhang, which is incorporated herein by reference in its entirety.

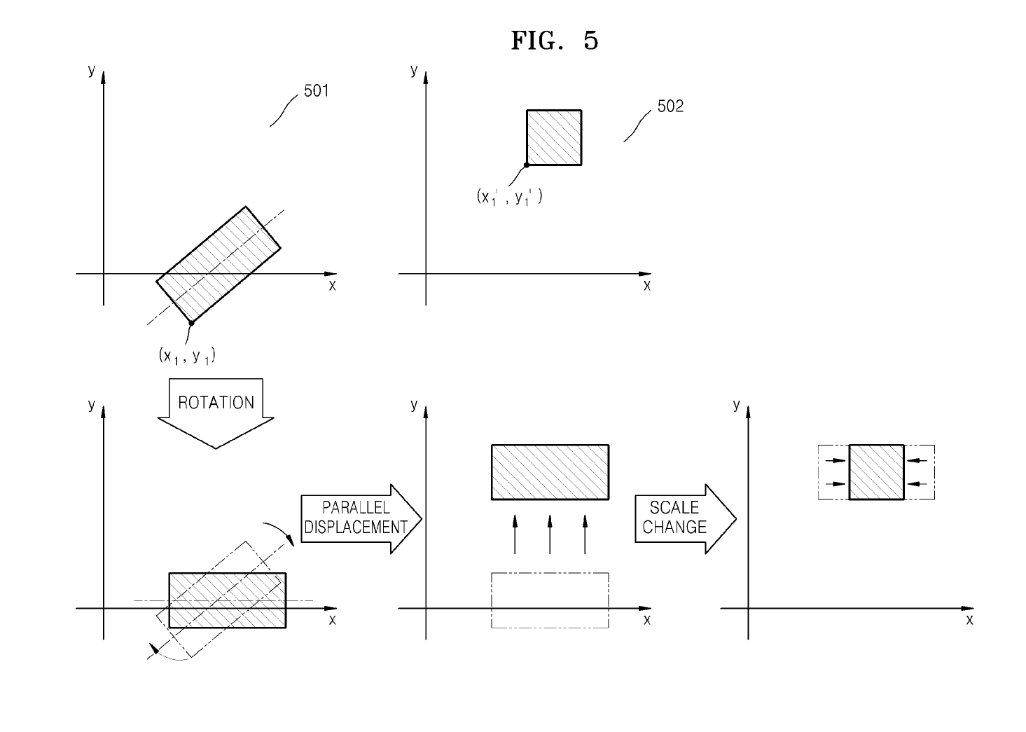

FIG. 5 illustrates a process of applying the affine transformation function in a 2-D image. A diagram 501 illustrates a state before applying the affine transformation, and a diagram 502 illustrates a state after applying the affine transformation. Although the rotation, the parallel displacement, and the scale control should be performed to apply the transformation, it is possible to determine coefficients of a matrix T_{affine} of the affine transformation function by obtaining first coordinates and last coordinates through the following Equation 9 in consideration that the affine transformation uses a one-to-one point correspondence.

$$[ \begin{matrix}{x}_{1}^{′} \\ {y}_{1}^{′}\end{matrix} ] = {T}_{affine} [ \begin{matrix}{x}_{1} \\ {y}_{1} \\ 1\end{matrix} ] = [ \begin{matrix}{a}_{1} & {b}_{1} & {c}_{1} \\ {a}_{2} & {b}_{2} & {c}_{2}\end{matrix} ] [ \begin{matrix}{x}_{1} \\ {y}_{1} \\ 1\end{matrix} ] \qquad ( 9 )$$

The affine transformation function is well known in the art, and therefore will not be described in detail here for conciseness.

The following Equation 10 is an equation for applying an affine transformation function obtained in three dimensions to each frame.

x_{ICP}(n)=T_{affine}(n)×x_{ASM}(n) (10)

Here, n is an integer indicating an n-th frame (1≦n≦N). x_{ASM}(n) indicates a landmark point vector in which the vector b that is the weight is changed in the image matching unit 204. x_{ICP}(n) includes location coordinate information of organ boundaries and internal structures in which a modification is reflected for each frame. It is possible to confirm a graphic figure of an organ with the naked eye if a voxel value corresponding to location coordinates is replaced or overlapped with a predetermined brightness value in an ultrasound image when matching the location coordinate information with the ultrasound image.

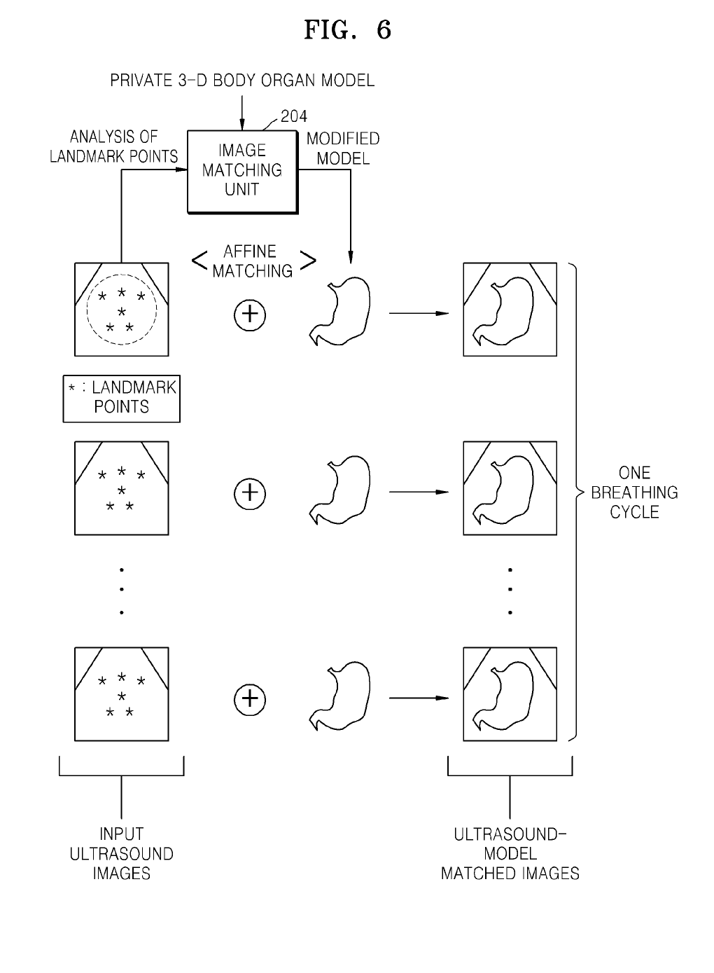

FIG. 6 illustrates a process of matching images performed by the image matching unit 204. FIG. 6 illustrate a process in which the image matching unit 204 matches a plurality of ultrasound images input during one breathing cycle to a private 3-D body organ model modified to reflect a change in an organ during breathing to generate a plurality of ultrasound-model matched images. In FIG. 6, the input ultrasound images are disposed in the left side of FIG. 6, and marks * in the input ultrasound images indicate landmark points. The input ultrasound images reflect various stages of breathing from inspiration to expiration.

A private 3-D body organ model generated by the private model generation unit 203 will be modified according to a change in an organ during breathing. However, a modification according to the breathing will be smaller than that due to diversity between individuals. Thus, when modifying the private 3-D body organ model according to a change in an organ during breathing, it may be faster and easier to adjust parameter values determined by the private model generation unit 203 compared to newly performing 3-D ASM algorithm. The affine transformation function T_{affine} is applied through the ICP algorithm by using a landmark point in which the modification has been reflected and a landmark point of an organ of the ultrasound image. Through the affine transformation, a size and a location of the private 3-D body organ model may be modified to be matched with a size and a location of an organ in the ultrasound image. Combining a modified model with the ultrasound image may be performed through a method of replacing or overlapping a pixel or voxel value of the ultrasound image corresponding to a location of a model with a predetermined value. A matched image is referred to as an ultrasound-model matched image and may be stored in the storage 207.

The image search unit 205 performs processes of a surgical operation. In the surgical operation, a graphic shape of an organ is output in an ultrasound image that is input in real time on a screen, and then a surgeon performs the surgical operation while confirming the graphic shape of the organ with the naked eye. Detailed operations of this process are as follow. First, a real time medical image of a patient is received. At this time, the real time medical image may be an image that is the same as that received by the image matching unit 204. Thus, for example, if a real time ultrasound image is received, by comparing the real time ultrasound image with medical images input to the image matching unit 204 during a predetermined period, an image that is most similar to the real time ultrasound image is determined, and an ultrasound-model matched image corresponding to the determined image is searched for in the storage 207, and then a found ultrasound-model matched image is output.

As an example in which the image search unit 205 searches for a similar image in the ultrasound image, there is a method of determining an image by detecting a location of the diaphragm. If a location of the diaphragm is X in the real time ultrasound image, the method involves searching for an image having the smallest difference by calculating a difference between the location X and a location of the diaphragm in each of the medical images input to the image matching unit 204 during the predetermined period.

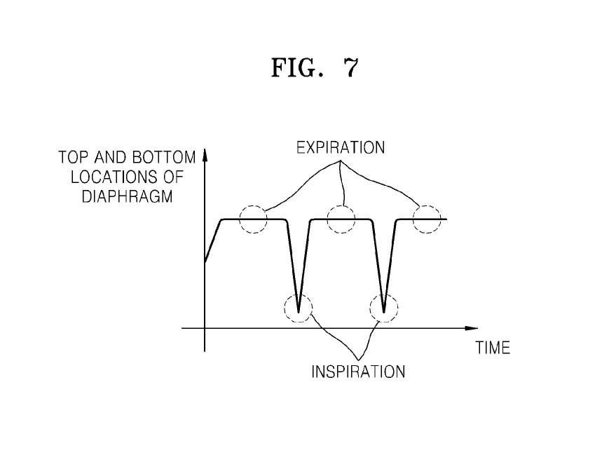

FIG. 7 is a graph illustrating an up and down movement of an absolute location of the diaphragm. On analyzing the graph, it is possible to confirm that the location of the diaphragm is regularly changed in a breathing cycle. A location of a probe 11 and a location of a patient may be fixed when capturing the medical images that are input to the image matching unit 204 during the predetermined period, and the real time medical image that is input to the image search unit 205. The reason is that a relative location of an organ in the image may be changed if the location of the probe 11 or the location of the patient is changed, and it is not possible to accurately and rapidly perform a search operation when comparing images if the relative location of the organ is changed.

As another example in which the image search unit 205 searches for a similar image in the ultrasound image, there is a method of determining an image by using a brightness difference between pixels. That is, this method involves using the fact that a brightness difference between the most similar images is the smallest. In greater detail, when searching for an image similar to an image (a second image) of a frame of the real time medical image among the medical images (first images) input during the predetermined period to use for matching, a brightness difference between pixels of one of the first images and pixels of the second image is calculated, and then a dispersion for the brightness difference is obtained. Next, brightness differences between pixels of the other images of the first images and pixels of the second image also are calculated and then dispersions for the brightness differences are obtained. Then, an image whose dispersion is the smallest may be determined as the most similar image.

The additional adjustment unit 206 may output an adjusted final result if a user adjusts the affine transformation function T_{affine} and the parameters of the 3-D ASM algorithm while viewing an output image. That is, the user may perform accurate transformation while viewing the output image with the naked eye.

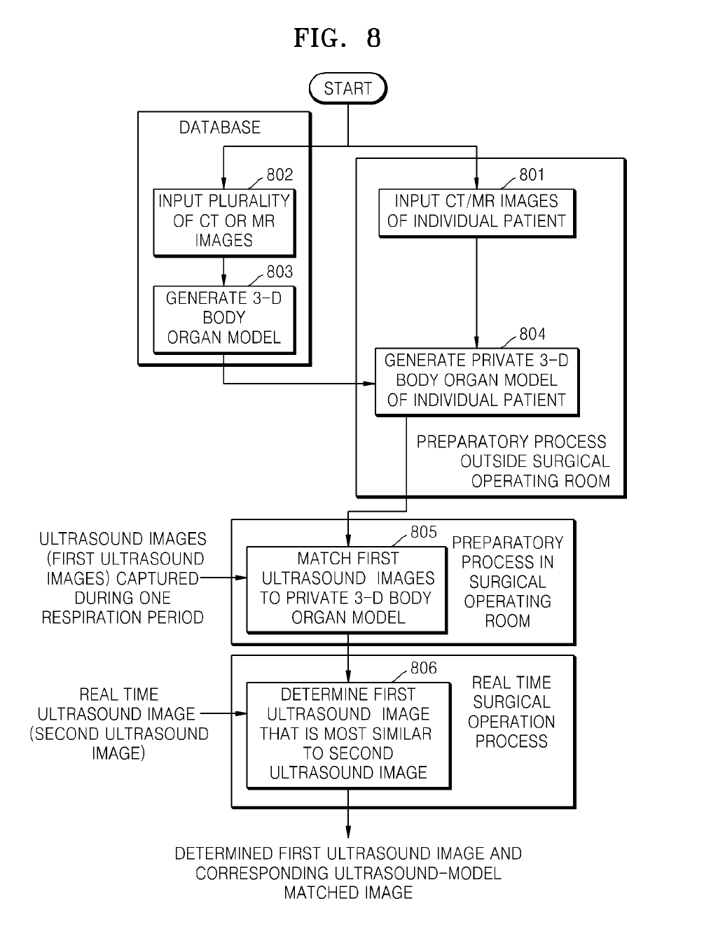

FIG. 8 is a flowchart illustrating a method of tracking a dynamic organ and a lesion based on a 3-D body organ model. Results of operations 802 and 803 may be stored in the medical image database (DB) 201 of FIG. 2. In the operation 802, CT or MR images for various breathing cycles of individuals are received. In the operation 803, a 3-D body organ model is generated based on the received images. At this time, as stated above, the 3-D ASM algorithm may be used.

In operation 801, a CT or MR image of an individual patient is received. In operation 804, the 3-D body organ model generated in the operation 803 is modified based on the received image of the individual patient. A process of generating the modified 3-D body organ model, namely a private 3-D body organ model, may be performed outside a surgical operating room as a preparatory process. In operation 805, ultrasound images (first ultrasound images) captured during one breathing cycle of a patient are received, and the first ultrasound images are matched to the private 3-D body organ model. A matched image is referred to as an ultrasound-model matched image, and may be stored in a temporary memory or in a storage medium such as storage 207 in FIG. 2. Operation 805 may be performed as a preparatory process in a surgical operating room. In operation 805, a location of the patient may be fixed. In addition, in operation 806, a location of a probe may be fixed. In operation 806, as a real operation in the surgical operation room, if an ultrasound image (a second ultrasound image) of the patient is input in real time, an image that is most similar to the second ultrasound image from among the first ultrasound images is determined, and then an ultrasound-model matched image corresponding to the determined first ultrasound image is output.

The various units 202, 204, 204, 205, and 206 in FIG. 2 may be implemented using hardware components and/or software components. Software components may be implemented by a processing device, which may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, a processing device may include multiple processors or a processor and a controller. In addition, different processing configurations are possible, such a parallel processors.

As used herein, a processing device configured to implement a function A includes a processor programmed to run specific software. In addition, a processing device configured to implement a function A, a function B, and a function C may include configurations, such as, for example, a processor configured to implement functions A, B, and C; a first processor configured to implement function A and a second processor configured to implement functions B and C; a first processor configured to implement functions A and B and a second processor configured to implement function C; a first processor to implement function A, a second processor configured to implement function B, and a third processor configured to implement function C; a first processor configured to implement functions A, B, C and a second processor configured to implement functions A, B, and C, and so on.

The software may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion.

In particular, the software and data may be stored by one or more non-transitory computer-readable storage mediums. The non-transitory computer-readable storage medium may include any data storage device that can store data that can be thereafter read by a computer system or processing device. Examples of a non-transitory computer-readable storage medium include read-only memory (ROM), random-access memory (RAM), CD-ROMs, magnetic tapes, floppy disks, and optical data storage devices. Also, functional programs, codes, and code segments for implementing the examples disclosed herein can be easily constructed by programmers skilled in the art to which the examples pertain based on and using the block diagram in FIG. 1 and the flow diagrams in FIGS. 2-7 and their corresponding descriptions as provided herein.

While this invention has been particularly shown and described with reference to various examples, it will be understood by those of ordinary skill in the art that various changes in form and details may be made in these examples without departing from the spirit and the scope of the invention as defined by the claims and their equivalents. The examples should be considered in a descriptive sense only and not for purposes of limitation. Therefore, the scope of the invention is defined not by the detailed description of the invention, but by the claims and their equivalents, and all variations falling within the scope of the claims and their equivalents are to be construed as being included in the invention.

What is claimed is:

1. A method of generating an image of an organ, the method comprising:
generating a three-dimensional (3-D) model of at least one organ of a patient based on a medical image of the at least one organ;
generating a plurality of matched images by matching a plurality of images showing a change of a shape of the at least one organ due to a body activity of the patient to the 3-D model of the at least one organ;
selecting one of the plurality of matched images based on a current body condition of the patient; and
outputting the selected matched image.

2. The method of claim 1, wherein the generating of the 3-D model comprises generating the 3-D model to show the shape of the at least one organ of the patient based on the medical image of the at least one organ.

3. The method of claim 2, wherein the selecting comprises selecting one of the plurality of matched images based a real time medical image showing the current body condition of the patient; and
the plurality of images and the real time medical image are ultrasound images.

4. The method of claim 1, wherein the generating of the plurality of matched images comprises:
modifying the 3-D model based on the change of the shape of the at least one organ; and
fitting a coordinate axis of the 3-D model to a coordinate axis of the plurality of images.

5. The method of claim 4, wherein the generating of the plurality of matched images further comprises generating the plurality of matched images by overlapping pixel or voxel values of the plurality of images with a predetermined brightness.

6. The method of claim 1, wherein the selecting comprises selecting one of the plurality of matched images corresponding to one of the plurality of images that is most similar to a real time medical image of the patient showing the current body condition of the patient.

7. The method of claim 6, wherein the selecting comprises:
calculating a difference between a location of a diaphragm in each of the plurality of images and a location of a diaphragm in the real time medical image; and
selecting one of the plurality of matched images that corresponds to one of the plurality of images for which the calculated difference is the smallest among all of the plurality of matched images.

8. The method of claim 1, wherein the generating of the 3-D model comprises:
extracting location coordinate information of a boundary and an internal structure of the at least one organ from the medical image;
designating coordinates of landmark points in the location coordinate information; and
generating a average 3-D model of the at least one organ based on the coordinates of the landmark points.

9. The method of claim 8, wherein the generating of the 3-D model further comprises changing the average 3-D model to a 3-D model reflecting a shape characteristic of the at least one organ of the patient.

10. The method of claim 9, wherein the generating of the 3-D model further comprises reflecting the shape characteristic of the at least one organ of the patient onto the medical image of the at least one organ.

11. The method of claim 10, wherein the shape characteristic comprises a shape and a location of a lesion of the at least one organ.

12. The method of claim 9, wherein the shape characteristic comprises a shape and a location of a lesion of the at least one organ.

13. The method of claim 8, wherein the extracting of the location coordinate information comprises determining a position in the medical image at which a change in a brightness value is a maximum as the location coordinate information of the boundary and the internal structure of the at least one organ.

14. The method of claim 8, wherein the extracting of the location coordinate information comprises determining a position in the medical image at which a frequency value of a discrete time Fourier transform (DTFT) is a maximum as the location coordinate information of the boundary and the internal structure of the at least one organ.

15. The method of claim 8, wherein the extracting of the location coordinate information comprises determining the location coordinate information of the boundary and the internal structure of the at least one organ based on coordinates input by a user.

16. The method of claim 1, wherein the plurality of images are images captured at predetermined intervals during a breathing cycle of the patient.

17. The method of claim 1, wherein the medical image of the at least one organ is an image captured using a computed tomography (CT) method.

18. The method of claim 1, wherein the medical image of the at least one organ is an image captured using a magnetic resonance (MR) method.

19. The method of claim 1, wherein the selecting comprises selecting one of the plurality of matched images based a real time medical image showing the current body condition of the patient; and
the plurality of images and the real time medical image are ultrasound images.

20. The method of claim 1, wherein the generating of the 3-D model comprises:
pre-generating the 3-D model prior to beginning preparations to treat the patient;
storing the pre-generated 3-D model in a database prior to beginning the preparations to treat the patient; and
retrieving the pre-generated 3-D model stored in the database as part of the preparations to treat the patient.

21. A non-transitory computer-readable storage medium storing a program for controlling a processor to perform the method of claim 1.

22. An apparatus for generating an image of an organ, the apparatus comprising:
an organ model generation unit configured to generate a 3-D model of at least one organ of a patient based on a medical image of the at least one organ;
an image matching unit configured to generate a plurality of matched images by matching a plurality of images showing a change of a shape of the at least one organ due to a body activity of the patient to the 3-D model of the at least one organ; and
an image search unit configured to select one of the plurality of matched images based on a current body condition of the patient, and output the selected matched image.

23. The apparatus of claim 22, further comprising an additional adjustment unit configured to further adjust the plurality of matched images according to an input from a user.

24. A method of generating an image of an organ, the method comprising:
generating an average three-dimensional (3-D) model of an organ based on a plurality of medical images of the organ;
generating a private 3-D model of the organ in a specific patient based on the average 3-D model of the organ and at least one medical image of the organ of the patient;
generating a plurality of matched images by matching a plurality of images of the organ of the patient in which a shape of the organ of the patient changes due to a body activity of the patient to the private 3-D model of the organ;
selecting one of the matched images based on a real time medical image of the organ of the patient reflecting a current body condition of the patient; and
outputting the selected matched image.
